(19) United States
(12) Patent Application Publication
(10) Pub. No.: US 2007/0250165 A1
(43) Pub. Date: Oct. 25, 2007

(54) Muscle-Polymer Constructs for Bone Tissue Engineering

(76) Inventors: Cato T. Laurencin, Earlysville, VA (US); Helen H. Lu, New York, NY (US); Michelle D. Kofron, Charlottesville, VA (US); Saadiq El-Amin, Charlottesville, VA (US); Mohamed A. Attawia, Canton, MA (US)

Correspondence Address: Licata & Tyrrell P.C., 66 E. Main Street, Marlton, NJ, 08053, US

(21) Appl. No.: 11/766,166

(22) Filed: Jun. 21, 2007

Related U.S. Application Data
(63) Continuation of application No. 10/467,400, filed on Jan. 28, 2004, now abandoned.
(60) Provisional application No. 60/270,191, filed on Feb. 21, 2001.

(51) Int. Cl. A61F 2/28

(52) U.S. Cl. 623/16.11

(57) ABSTRACT

Bone grafting materials containing a polymer scaffold loaded with bone morphogenetic proteins and populated with muscle cells induced by the bone morphogenetic proteins to exhibit an osteoblastic phenotype and to synthesize bone tissue are provided. Also provided are methods for using these polymer scaffolds in bone grafting procedures.

This patent application is a continuation of U.S. patent application Ser. No. 10/467,400 filed Jan. 28, 2004 which claims the benefit of priority from U.S. Provisional Application Ser. No. 60/270,191 filed Feb. 21, 2001, teachings of each of which are hereby incorporated by reference in their entirety.

This invention was sponsored in part by the National Science Foundation (Grant Number BES9553162/BES981782). The U.S. government may therefore have certain rights in the invention.

### FIELD OF THE INVENTION

The present invention relates to polymer scaffolds for use in surgical bone repair and replacement. The scaffold is pre-loaded with bone morphogenetic proteins (BMPs) which induce muscle cells to exhibit an osteoblastic phenotype and to synthesize bone tissue. Under controlled culturing conditions, it has been found that the BMP-polymer constructs support the attachment, growth and differentiation of muscle cells into osteoblast-like cells. After sufficient bone tissue has formed ex vivo, the cultured scaffold can then be implanted into a patient.

### BACKGROUND OF THE INVENTION

Over one million bone repair operations are performed in the U.S. every year, with autogenic bone grafting being the clinical standard in surgical bone repair and replacement. Despite a clinical success rate of 80-90%, shortcomings associated with this procedure include a second operation in order to obtain the graft, the limited supply of autogenous bone, architectural constraints and potential donor site morbidity. Thus, other bone grafting materials are needed.

Recently, bone tissue engineering has emerged as an alternative grafting procedure, where a biocompatible scaffold is populated and maintained with autogenous cells ex vivo and later implanted into the body after sufficient bone tissue has been formed. In this approach, the patient's bone cells, usually obtained through bone biopsies are used. However, the biopsy can be difficult and painful for the patient, and only a limited amount of bone can be procured in this strategy.

The three main factors that govern the success of tissue engineered bone are the matrix, the cellular component, and the incorporation of bioactive molecules. The scaffold is often constructed from the synthetic polymers polylactide (PLA), polyglycolide (PGA) and their co-polymers (PLAGA). The biocompatibility of these polymers is well documented, and they have been approved by the Food and Drug Administration and are used clinically as surgical sutures and fixation devices.

Scaffolds made from biodegradable polymers and loaded with bone morphogenetic proteins (BMPs) have also been described in the literature. The cellular component of these scaffolds was either pluripotent stem cells, osteoblasts or chondrocytes. Like bone cells, however, these types of cells are difficult to harvest, with the procedures being often very painful and traumatic to the host.

### SUMMARY OF THE INVENTION

An object of the present invention is to provide a bone grafting material comprising a polymer scaffold loaded with bone morphogenetic proteins and populated with muscle cells induced by the bone morphogenetic proteins to exhibit an osteoblastic phenotype and to synthesize bone tissue.

Another object of the present invention is to provide methods for using these polymer scaffolds in bone grafting procedures.

### DETAILED DESCRIPTION OF THE INVENTION

The present invention relates to a bone grafting material for use in surgical bone repair and replacement. The bone grafting material of the present invention comprises a scaffold, preferably a polymer scaffold, pre-loaded with bone morphogenetic proteins (BMPs) and populated with muscle cells. It has now been found that BMPs induce the muscle cells of the scaffold to exhibit an osteoblastic phenotype and to synthesize bone tissue. Unlike osteoblasts and other cells used in the prior art to populate polymer scaffolds, muscle cells are more readily available, and are obtainable via a simple subcutaneous procedure that is less painful and traumatic for the patient. Muscle tissue makes up 48% of total body mass, ensuring a sufficient supply of cells. An additional advantage of this approach is the elimination of donor site morbidity, which has hindered the success of autogenous bone grafts.

The feasibility of using these muscle-polymers constructs in bone tissue engineering was demonstrated under controlled culturing conditions. For these experiments, the polymer component of the scaffold, poly(lactic-co-glycolide) was selected because of its documented degradability and biocompatibility. However, as will be understood by those of skill in the art upon reading this disclosure, other polymers known in the art for use as polymer scaffolds can also be used. Examples of polymers useful in the scaffolds of the present invention include, but are not limited to, lactic acid polymers such as poly(L-lactic acid (PLLA), poly(DL-lactic acid (PLA), and poly(DL-lactic-co-glycolic acid)(PLGA) and co-polymers thereof, polyorthoesters, polyanhydrides, polyphosphazenes, polycaprolactones, polyhydroxybutyrates, degradable polyurethanes, polyanhydrideco-imides, polypropylene fumarates, and polydiaxonane.

BMPs were then incorporated into the polymer scaffold, as these proteins play an important role in osteogenesis. In vitro, these polymer-BMP scaffolds were found to support the attachment, growth and differentiation of quadriceps and triceps muscle cells into osteoblast-like cells, and resulted in the formation of mineralized tissue.

More specifically, thin film discs of poly(lactic-co-glycolide)(PLAGA), with and without BMP-7, were fabricated using a traditional solvent-casting method. In this process, the polymer was first dissolved in methylene chloride, then poured into a Teflon-coated dish. Reconstituted human recombinant BMP-7 was slowly mixed into the polymer solution. The dishes were then placed in a −20° C. freezer to allow solvent evaporation. The thin film matrices containing BMP (PLAGA-BMP) were subsequently bored into 1.0 cm diameter discs. PLAGA discs without BMP-7 and tissue culture plastic served as control groups.

Muscle cells were isolated from the triceps and quadriceps muscles of 1 kg New Zealand White Rabbits. The cells were grown to confluence, then seeded onto the discs at a density of 50,000 cells/scaffold. The cells were cultured on the discs in vitro in a 37° C. and 5% CO₂ environment, using HAM F-12+10% Fetal Bovine Serum as a nutrient source. Mineralization medium, containing ascorbic acid and β-glycerol phosphate, was used after seven days.

At specific time points, scanning electron microscopy (SEM) was used to verify the triceps and quadriceps muscle cells attachment, growth and cellular morphology upon the scaffolds. Energy dispersive x-ray analysis (EDXA) was used to examine mineral formation. By day 18, EDXA detected significantly higher levels of phosphorous and calcium, the major mineral components of bone, on the PLAGA-BMP discs cultured with rabbit triceps cells. The corresponding control discs without BMP failed to produce comparable mineral levels.

The muscle cells expressed classic markers for the osteoblastic phenotype, specifically, osteocalcin, alkaline phosphatase, and most importantly, the formation of mineralized tissue. The production of osteocalcin was imaged using immunofluorescence microscopy. Synthesis of mineralized tissue by the muscle cells was quantified using Alizarin Red staining following an assay by Jacobs, et al.

Thus, as demonstrated herein, scaffolds pre-loaded with bone morphogenetic proteins (BMPs) can be used to induce muscle cells to exhibit the osteoblastic phenotype. These polymer-BMP scaffolds supported the attachment, growth and differentiation of muscle cells into osteoblast-like cells, and resulted in the formation of mineralized tissue.

Accordingly, the polymers scaffolds loaded with BMPs and populated with muscle cells induced to exhibit an osteoblastic phenotype provide a useful bone grafting material for implantation in surgical bone repair and replacement. In these procedures, the BMP loaded scaffold is populated and maintained with autogenous muscles cells ex vivo and later implanted into the body after sufficient bone tissue has been formed. Methods for implantation of such materials into a patient in need thereof are well known and used routinely by those of skill in the art.

What is claimed is:

1. A bone grafting material comprising a polymer scaffold loaded with bone morphogenetic proteins and populated with muscle cells induced by the bone morphogenetic proteins to exhibit an osteoblastic phenotype and to synthesize bone tissue.

2. A method for using the bone grafting material of claim 1 in a bone grafting procedure comprising maintaining the material ex vivo until sufficient bone tissue has been formed and implanting the material into a patient in need thereof.
